(19) United States
(12) Patent Application Publication
(10) Pub. No.: US 2024/0344141 A1
(43) Pub. Date: Oct. 17, 2024

(54) CELL-FREE DNA ANALYSIS IN THE DETECTION AND MONITORING OF PANCREATIC CANCER USING A COMBINATION OF FEATURES

(71) Applicant: ClearNote Health, Inc., San Diego, CA (US)

(72) Inventors: Samuel LEVY, San Diego, CA (US); Anna BERGAMASCHI, San Diego, CA (US); David HAAN, San Diego, CA (US); Gulfem Dilek GULER, San Diego, CA (US); Wayne VOLKMUTH, San Diego, CA (US)

(21) Appl. No.: 18/614,553

(22) Filed: Mar. 22, 2024

Related U.S. Application Data
(60) Provisional application No. 63/491,738, filed on Mar. 22, 2023.

(51) Int. Cl. C12Q 1/6886; G16B 20/20; G16H 50/30

(52) U.S. Cl. CPC C12Q 1/6886; G16B 20/20; G16H 50/30; C12Q 2600/154; G01N 2800/50

(57) ABSTRACT

The present invention provides a method for detecting, assessing, and monitoring pancreatic cancer without need for a surgical biopsy or other invasive means.

### CROSS-REFERENCE TO RELATED APPLICATION

This application claims priority under 35 USC § 119 (e) to Provisional U.S. Patent Application Ser. No. 63/491,738, filed 22 Mar. 2023, the disclosure of which is incorporated herein by reference in its entirety.

### INCORPORATION BY REFERENCE: “20230322 3599-0016P MODEL” AS TABLE 1

A table entitled “20230322 3599-0016P Model” is electronically submitted herewith as an ASCII plain text file pursuant to the provisions of 37 CFR § 1.58 (c) through (e) and is incorporated by reference herein in its entirety. The file was created on Mar. 22, 2023 at 6:42 AM and is 4.2 MB in size, and is referred to hereinafter as “Table 1.”

LENGTHY TABLES
The patent application contains a lengthy table section. A copy of the table is available in electronic form from the USPTO web site (https://seqdata.uspto.gov/?pageRequest=docDetail&DocID=US20240344141A1). An electronic copy of the table will also be available from the USPTO upon request and payment of the fee set forth in 37 CFR 1.19(b)(3).

### BACKGROUND OF THE INVENTION

Cancer is the second leading cause of death globally. Cancer mortality is exacerbated by diagnosis at late stage when prognosis is poor. Earlier cancer detection offers the opportunity to improve patient outcomes by identifying tumors when treatment is more likely to be effective. While breast, colorectal and lung cancers are among the few cancers for which screening modalities exist, screening tests that are currently used in the clinic can be expensive, invasive, and limited to detection of a single cancer type; this, in turn, may necessitate multiple tests, further increasing the cost for overall early cancer detection and resulting in possible delay of treatment. Liquid biopsy-based multi-cancer early detection tests aim to address these limitations and complement these screening approaches.

Current non-invasive methods for early cancer detection rely upon genetic, epigenetic, or proteomic changes in cell-free DNA (cfDNA) that is obtained from plasma or in exosomes circulating in blood. While these methods can achieve a certain level of performance for cancer detection and therapy response prediction, there is an ongoing need to improve the performance of non-invasive tests to detect more cancers earlier (i.e., to increase sensitivity) and distinguish non-cancer cases from being identified falsely as positive cases (i.e., to increase specificity).

Pancreatic cancer (PaC) is the fourth leading cause of cancer deaths in the United States (1). To date, there are no molecular early-detection tools available for PaC (2). Poor survival outcomes in PaC are largely attributable to the late-stage diagnosis of the disease for the majority (87%) of subjects, including distant metastasis in 49% (1). Late-stage diagnosis deprives individuals of potentially curative interventions, such as surgery (3), and negatively impacts survival rates, with 5-year survival probability reduced more than 10-fold, from 38% to 3%, for localized compared with distant metastatic disease (1). It is therefore evident that early diagnosis is paramount for better survival outcomes in subjects with PaC.

Epigenetic control of DNA state and chromatin regulation is known to underpin cancer onset and progression (4, 5), and interest in the assessment of epigenetic profiles for tumor detection and characterization has increased in recent years (6).

5-Hydroxymethylcytosine (5hmC) is a stable epigenetic marker that arises as the first step of active demethylation of the cytosine base in DNA by ten-eleven translocation enzymes (also known as TET enzymes), marking regions of active transcription and gene regulation (7). 5hmC is positively correlated with gene expression and regulation in multiple biological contexts (8-10). As such, 5hmC profiles have yielded distinctive signatures that enable definition of tissue identity and cellular states (9, 11) and 5hmC is a valuable marker to identify the tissue of origin. Notably, 5hmC profiling has been used to detect cancer in cfDNA in the plasma of individuals with cancers, including pancreatic, lung, hepatocellular, colon, and gastric cancers (12-14).

While there are certain genetic syndromes associated with an increased risk of developing PaC (e.g., Peutz-Jeghers syndrome or germline mutations in CDKN2A, BRCA2, or PALB2), and for which surveillance is recommended (15, 16), there are other well-recognized risk factors for PaC that are not covered in surveillance and management guidelines. More than 50% of subjects diagnosed with PaC have a prior diagnosis of diabetes (17). Furthermore, while subjects with diabetes have a 1.5- to 2-fold increased risk of developing PaC compared with the general population (18), the risk is increased 6- to 8-fold in people aged≥50 years who are diagnosed with new-onset (≤3 years previously) type 2 diabetes (NOD) (19). Indeed, nearly 25% of all new diagnoses of PaC in the United States are identified in subjects with NOD (20). Surveillance of the NOD population for signs of PaC therefore presents an opportunity to shift PaC diagnosis to earlier stage disease, thereby improving outcomes through timely intervention.

There is an ongoing need in the art for the development and validation of a novel, noninvasive cfDNA-based method for the detection, assessment, and monitoring of pancreatic cancer and can be deployed in the clinical setting. More particularly, there is an ongoing need to drastically improve the survival rates associated with pancreatic cancer by providing a method for reliably detecting pancreatic cancer in its early stages.

### SUMMARY OF THE INVENTION

Accordingly, the present invention provides a method for detecting, assessing, and monitoring pancreatic cancer without need for a surgical biopsy or other invasive means. The method is a “liquid biopsy” based technique that relies on an analysis involving a consideration of multiple feature types, which include, without limitation, two or more of:

An epigenetic signature, particularly a 5-hydroxymethylcytosine signature;
5hmC-containing fragment counts in annotated CpG-island genomic regions;
5hmC-containing fragment counts in CTCF-binding regions;
5hmC-containing fragment counts in annotated enhancer regions;
5hmC-containing fragment counts in annotated gene body regions;
5hmC-containing fragment counts in annotated 3′-UTR genomic regions;
WGS fragment counts in each of a series of windows along the genome and/or WGS fragment counts in each of a set of size range bins;
WGS fragment counts in 100 kb genomic regions for determination of copy number variation (CNV);
Epigenetic features other than 5hmC, e.g., 5-methylcytosine (5mC)-related features such as 5mC location, level, and or fragment size;
cfDNA concentration in plasma;
Plasma level of CA 19-9;
Histone markers such as H3K4me1, H3K4me3, H3K9me3, H3K27ac, H3K27me3 and H3K36me3;
Other biomarkers not expressly included within the above categories; and
Any patient specific clinical parameter that tends to correlate with the risk of pancreatic cancer.

With respect to the latter category, clinical features may also be taken into account in the present method in either or both of two ways. First, one or more patient-specific clinical parameter may be used to exclude or include samples from certain patients (e.g., cigarette smokers, individuals under 35, presence of pancreatitis, etc.). Alternatively, such a parameter may be incorporated into the present analysis as feature type. Examples of patient-specific clinical features include, by way of illustration and not limitation, lesion size; lesion grade; lesion stage; lesion location; presence or absence of pancreatic inflammation; presence or absence of jaundice; presence or absence of diabetes, including Type I and Type II diabetes; presence or absence of other pathologies or symptoms; pro-inflammatory cytokine levels; patient age; patient weight; patient BMI; patient gender; patient ethnicity; family history; physical activity; diet; cigarette smoking status; and exposure or lack of exposure to a known carcinogen.

The method of the invention involves, ultimately, calculating a probability score indicating the likelihood that a patient has cancer. In contrast to prior attempts to detect the presence of pancreatic cancer from a blood sample, the present method is precise and efficient, exhibits excellent specificity and sensitivity, and can be carried out on very small samples, e.g., on a cfDNA sample of 10 ng or less.

Briefly, that process involves counting cfDNA fragments having certain characteristics, e.g., 5hmC-containing fragments only, 5hmC-containing fragments that map to a specific location on the genome, DNA fragments generated in a 5hmC assay or using WGS, and the like, as will be explained in detail elsewhere herein. The fragment counts are used in a count vector for a given set of features (e.g., promoters), which is normalized and then scored using each of a plurality of base models. The base models are created as a (penalized) logistic regression fit, meaning that one score is generated for each base model by summing the product of the correlation coefficient x the cpm values (or coefficient x cpm x log 1+conc).

Ultimately, it is these base model scores (one per base model per sample, calculated as just described), rather than individual count data, that are input into an ensemble model, e.g., a stacked ensemble model, to generate an ensemble score. Table 1, incorporated by reference herein, sets forth a number of base models and an ensemble model used in the present method, as follows:
5hmC fragment counts in annotated CpG-Island genomic regions, decontaminated, CPM-normalized, glmnet fit, entitled “CpG-island_decontaminated_CPM_GLMNET” (the term “decontaminated” refers to a computational method employed to remove noise in 5hmC data introduced by the presence of non-5hmC fragment in the 54mC assay);
5hmC fragment counts in annotated CTCF-binding genomic regions, decontaminated, CPM-normalized, glmnet fit, entitled “CTCF_decontaminated_CPM_GLMNET”;
5hmC fragment counts in annotated enhancer genomic regions, decontaminated, cfDNA plasma concentration interaction term transformed, CPM-normalized, glmnet fit, entitled “enhancer_plasmaconc_inter_CPM_GLMNET”;
5hmC fragment counts in annotated gene body genomic regions, decontaminated, cfDNA plasma concentration interaction term transformed, CPM-normalized, glmnet fit, entitled “gene_plasmaconc_inter_CPM_GLMNET”;
5hmC fragment counts in annotated promoter genomic regions, decontaminated, CPM-normalized, glmnet fit, entitled “promoter_plasmaconc_inter_CPM_GLMNET”;
5hmC fragment counts in annotated 3′UTR genomic regions, decontaminated, CPM-normalized, glmnet fit, entitled “three_prime_UTR_decontaminated_CPM_GLMNET”;
WGS fragment counts in 2 MB genomic regions, divided by fragments length ranges, mean-centered and scaled, glmnet fit, entitled “Frag_Data_2 MB_SML_scaled_GLMNET”;
WGS fragment counts in 100 kb genomic regions, CPM-normalized, glmnet fit, entitled “WGS_CNV_100 kb_CPM_GLMNET”; and
ENSEMBLE, with the above base model predictions combined and using a glmnet fit.

In one embodiment, then, the invention provides a method for detecting the likelihood of pancreatic cancer in a patient from a blood sample obtained from the patient, the method comprising:
(a) extracting a cell-free DNA (cfDNA) sample from the blood sample;
(b) counting 5-hydroxymethylcytosine (5hmC)-containing DNA fragments in the cfDNA sample to give a total 5hmC fragment count;
(c) counting 5hmC-containing DNA fragments that map to a first genomic location, giving a first genomic location 5hmC fragment count;
(d) counting 5hmC-containing DNA fragments that map to a second genomic location, giving a second genomic location 5hmC fragment count;
(e) optionally carrying out (d) to count 5hmC-containing DNA fragments that map to at least one additional genomic location, giving at least one additional genomic 5hmC fragment count;
(f) normalizing the 5hmC fragment counts obtained in (b) through (e) to give normalized 5hmC fragment counts;
(g) scoring the normalized 5hmC fragment counts using each of a plurality of base models each comprising a penalized logistic regression fit, wherein one score is generated for each base model by summing the product of a determined correlation coefficient times the normalized 5hmC fragment count, thereby providing a plurality of base model scores; and
(h) inputting the plurality of base model scores into an ensemble model to generate a final score in the form of a probability score p between zero and 1 indicating the likelihood of pancreatic cancer in the patient.

In another embodiment, the method further includes, in addition to the 5hmC-containing DNA fragment counts, incorporation of at least one additional feature type in the ensemble model of (h). Examples of additional feature types include WGS fragment counts in each of a series of windows along the genome and/or WGS fragment counts in each of a set of size range bins; copy number variation; epigenetic feature information other than 5hmC-related information, e.g., 5-methylcytosine (5mC) levels and/or 5mC-containing cfDNA fragment counts; CA 19-9 level; histone marker information; cfDNA concentration in the blood sample; and any one of a number of patient-specific clinical parameters that correlate with the risk of pancreatic cancer, as discussed elsewhere herein.

In a further embodiment, the invention provides a a method for detecting the likelihood of pancreatic cancer in a patient from a blood sample obtained from the patient, the method comprising:
(a) extracting a cell-free DNA (cfDNA) sample from the blood sample;
(b) counting 5-hydroxymethylcytosine (5hmC)-containing DNA fragments in the cfDNA sample to give a total 5hmC fragment count;
(c) determining 5hmC-containing fragment counts in CTCF-binding regions, annotated enhancer regions, annotated gene body regions, and annotated 3′-UTR genomic regions, to provide individual location 5hmC fragment counts;
(d) determining WGS fragment counts in each of a series of genomic windows along the genome and/or WGS fragment counts in each of a set of size range bins;
(e) determining WGS fragment counts in specifically sized genomic regions for determination of copy number variation (CNV); (f) determining cfDNA concentration in the blood sample;
(f) normalizing the fragment counts obtained in (b) through (e) to give normalized fragment counts;
(g) scoring the normalized fragment counts using each of a plurality of base models each comprising a penalized logistic regression fit, wherein one score is generated for each base model by summing the product of a determined correlation coefficient times the normalized fragment count, thereby providing a plurality of base model scores; and
(h) inputting the plurality of base model scores into an ensemble model to generate a final score in the form of a probability score p between zero and 1 indicating the likelihood of pancreatic cancer in the patient.

In a related embodiment, the aforementioned method further comprises incorporating at least one additional feature type in the ensemble model of (h). In one aspect of this embodiment, the at least one additional feature type comprises cfDNA concentration in the blood sample.

In a further embodiment, the invention provides a method for monitoring changes in a patient's pancreatic cancer state, comprising repeating any of the above-described processes at intervals throughout an extended monitoring period and determining differences in probability score over time.

### BRIEF DESCRIPTION OF THE DRAWINGS

FIG. 1 schematically illustrates 5hmC enrichment, WGS features, and machine-learning workflow to develop and validate a predictive model for detection of pancreatic cancer.

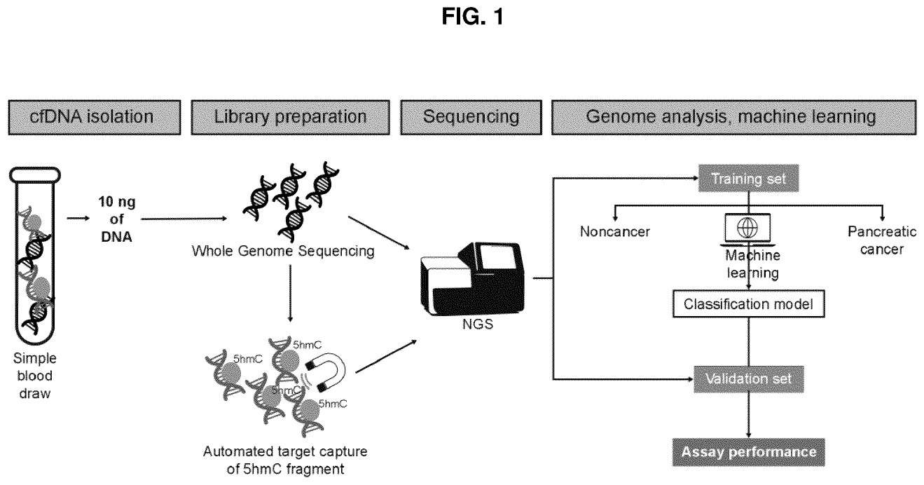

FIGS. 2A and 2B identify characteristics of the training and validation datasets, with FIG. 2A indicating the samples included in the training and validation datasets and FIG. 2B illustrating the distribution of pancreatic cancer risk factors in the training and validation datasets. FIG. 2C shows the correlation found between 5hmC fold change over gene body features included in the model used in the Example, comparing non-diabetes PaC (Y-axis) and NOD PaC (X-axis) to corresponding noncancer samples. The linear regression equation and Pearson correlation with associated P values are reported in the panel.

FIG. 3 provides the results of the clinical concordance analysis between CA19-9 and the PaC detection algorithm used herein.

FIGS. 4A and 4B provide the results of the analysis of hydroxymethylated genes in cfDNA, with FIG. 4A providing the mean CPM values in cfDNA of 366 hypo-hydroxymethylated genes and FIG. 4B providing the mean CPM values in cfDNA of 43 hyper-hydroxymethylated genes differentially expressed between pancreatic cancer and normal pancreatic tissues. Each data point represents a subject and corresponding mean CPM count of either 366 (FIG. 4A) or 43 (FIG. 4B) genes.

FIGS. 5A, 5B, 5C, and 5D pertain to model training. FIG. 5A shows the ROC curves from predictive modeling using regularized regression model (elastic net) on the training dataset with 132 pancreatic cancer and 528 noncancer cfDNA samples. FIG. 5B provides the ROC curves for each of four disease stages. FIG. 5C gives the pancreatic cancer prediction score by disease stage, with the dashed line indicating the 98% specificity threshold. FIG. 5D indicates the mean sensitivity range (62.8-67.4%) and specificity range (97.5-98.1%) across 10-fold cross validation, repeated for 10 iterations.

FIGS. 6A and 6B pertain to the 5hmC and WGS features included in the PaC prediction model, with FIG. 6A providing the total number and FIG. 6B indicating the relative proportions of features used to classify PaC versus noncancer.

FIG. 7 is a table providing a summary of demographics and disease status for the subjects evaluated in the Example.

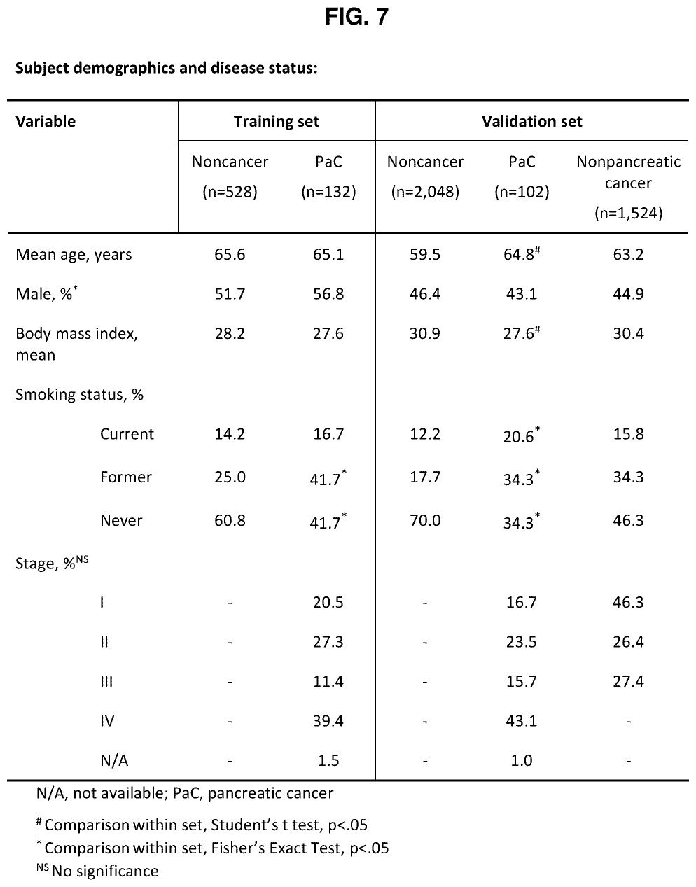

FIG. 8 is a table indicating the sensitivity and specificity observed with pancreatic cancer and noncancer in the training and validation datasets.

### DETAILED DESCRIPTION OF THE INVENTION

### I. Definitions and Terminology

Unless defined otherwise, all technical and scientific terms used herein have the meaning commonly understood by one of ordinary skill in the art to which the invention pertains. Specific terminology of particular importance to the description of the present invention is defined below. Other relevant terminology is defined in International Patent Publication No. WO 2017/176630 to Quake et al. for “Noninvasive Diagnostics by Sequencing 5-Hydroxymethylated Cell-Free DNA.” The aforementioned patent publication as well as all other patent documents and publications referred to herein are expressly incorporated by reference.

In this specification and the appended claims, the singular forms “a,” “an” and “the” include plural referents unless the context clearly dictates otherwise.

Numeric ranges are inclusive of the numbers defining the range. Unless otherwise indicated, nucleic acids are written left to right in 5′ to 3′ orientation; amino acid sequences are written left to right in amino to carboxy orientation, respectively.

The abbreviations used herein are as follows: 5hmC, 5-hydroxymethylcytosine; auROC, area under the receiver operating characteristic curve; CA19-9, carbohydrate antigen 19-9; cfDNA, cell-free DNA; CI, confidence interval; CNV, copy number variation; CPM, counts per million; gDNA, genomic DNA; IPMN, intraductal papillary mucinous neoplasm; IRB, Institutional Review Board; N/A, not available; NOD, new-onset diabetes; NS, not significant; PaC, pancreatic cancer; T2D, type 2 diabetes; WGS, whole genome sequencing.

The headings provided herein are not limitations of the various aspects or embodiments of the invention. Accordingly, the terms defined immediately below are more fully defined by reference to the specification as a whole.

The term “sample” as used herein relates to a material or mixture of materials, typically in liquid form, containing one or more analytes of interest. The biological samples evaluated herein are blood samples obtained from a patient.

A “nucleic acid sample” as that term is used herein refers to a biological sample comprising nucleic acids. The nucleic acid sample may be a genomic DNA sample, or it may be comprised of cfDNA wherein the sample is substantially free of histones and other proteins, such as will be the case following cfDNA purification.

A “sample fraction” refers to a subset of an original biological sample, and may be a compositionally identical portion of the biological sample, as when a blood sample is divided into identical fractions. Alternatively, the sample fraction may be compositionally different, as will be the case when, for example, certain components of the biological sample are removed, with extraction of cell-free nucleic acids being one such example.

As used herein, the term “cell-free nucleic acid” encompasses both cfDNA and cfRNA, where the cfDNA and cfRNA may be in a cell-free fraction of a biological sample comprising a body fluid. The body fluid may be blood, including peripheral blood, serum, or plasma. In most instances, the biological sample is a blood sample, and a cell-free nucleic acid sample, e.g., a cell-free DNA sample, is extracted therefrom using now-conventional means known to those of ordinary skill in the art and/or described in the pertinent texts and literature; kits for carrying out cell-free nucleic acid extraction are commercially available (e.g., the AllPrep® DNA/RNA Mini Kit and QIAmp DNA Blood Mini Kit, both available from Qiagen, or the MagMAX Cell-Free Total Nucleic Acid Kit and the MagMAX DNA Isolation Kit, available from ThermoFisher Scientific). Also see, e.g., Hui et al. Fong et al. (2009) Clin. Chem. 55 (3): 587-598.

The term “adapter-ligated,” as used herein, refers to a nucleic acid that has been ligated to an adapter. The adapter can be ligated to a 5′ end and/or a 3′ end of a nucleic acid molecule. As used herein, the term “adding adapter sequences” refers to the act of adding an adapter sequence to the end of fragments in a sample. This may be done by filling in the ends of the fragments using a polymerase, adding an A tail, and then ligating an adapter comprising a T overhang onto the A-tailed fragments. Adapters are usually ligated to a DNA duplex using a ligase, while with RNA, adapters are covalently or otherwise attached to at least one end of a cDNA duplex preferably in the absence of a ligase.

The term “amplifying” as used herein refers to generating one or more copies, or “amplicons,” of a template nucleic acid, such as may be carried out using any suitable nucleic acid amplification technique, such as technology, such as PCR, NASBA, TMA, and SDA.

The terms “enrich” and “enrichment” refer to a partial purification of template molecules that have a certain feature (e.g., nucleic acids that contain 5-hydroxymethylcytosine) from analytes that do not have the feature (e.g., nucleic acids that do not contain hydroxymethylcytosine). Enrichment typically increases the concentration of the analytes that have the feature by at least 2-fold, at least 5-fold or at least 10-fold relative to the analytes that do not have the feature. After enrichment, at least 10%, at least 20%, at least 50%, at least 80% or at least 90% of the analytes in a sample may have the feature used for enrichment. For example, at least 10%, at least 20%, at least 50%, at least 80% or at least 90% of the nucleic acid molecules in an enriched composition may contain a strand having one or more hydroxymethylcytosines that have been modified to contain a capture tag.

The term “sequencing,” as used herein, refers to a method by which the identity of at least 10 consecutive nucleotides (e.g., the identity of at least 20, at least 50, at least 100 or at least 200 or more consecutive nucleotides) of a polynucleotide is obtained.

The terms “next-generation sequencing” (NGS) or “high-throughput sequencing”, as used herein, refer to the so-called parallelized sequencing-by-synthesis or sequencing-by-ligation platforms currently employed by Illumina, Life Technologies, Roche, etc. Next-generation sequencing methods may also include nanopore sequencing methods such as that commercialized by Oxford Nanopore Technologies, electronic detection methods such as Ion Torrent technology commercialized by Life Technologies, and single-molecule fluorescence-based methods such as that commercialized by Pacific Biosciences.

The term “read” as used herein refers to the raw or processed output of sequencing systems, such as massively parallel sequencing. In some embodiments, the output of the methods described herein is reads. In some embodiments, these reads may need to be trimmed, filtered, and aligned, resulting in raw reads, trimmed reads, aligned reads.

A “Unique Molecular Identifier” (UMI) refers to a relatively short nucleic acid sequence that is appended to every nucleic acid template molecule in a sample, and is random, such that, providing that the UMI sequence is of sufficient length, every nucleic acid template molecule is attached to a unique UMI sequence. UMI sequences, as is known in the art, can be used to account for and offset amplification and sequencer errors, allow a user to track duplicates and remove them from downstream analysis, and enable molecular counting, and, in turn, the determination of an analyte concentration. See, e.g., Casbon et al. (2011) Nuc. Acids Res. 39 (12): 1-8. The “unique molecule” here is the identity of the nucleic acid template molecules.

In some embodiments, a UMI may have a length in the range of from 1 to about 35 nucleotides, e.g., from 3 to 30 nucleotides, 4 to 25 nucleotides, or 6 to 20 nucleotides. In certain cases, the UMI may be error-detecting and/or error-correcting, meaning that even if there is an error, then the code can still be interpreted correctly. The use of error-correcting sequences is described in the literature (e.g., in U.S. Patent Publication Nos. U.S. 2010/0323348 to Hamati et al. and U.S. 2009/0105959 to Braverman et al., both of which are incorporated herein by reference).

A “barcode” is also a short nucleic acid sequence, but a single barcode is appended to each DNA molecule in a sample, thereby serving to identify the sample of origin following processing, amplification, and sequencing of a group of combined samples.

The term “detection” is used interchangeably with the terms “determining,” “measuring,” “evaluating,” “assessing,” “assaying,” and “analyzing,” to refer to any form of measurement, and include determining if an element is present or not. These terms include both quantitative and/or qualitative determinations. Assessing may be relative or absolute. “Assessing the presence of” thus includes determining the amount of a moiety present, as well as determining whether it is present or absent. Assessing the level at a hydroxymethylation biomarker locus refers to a determination of the degree of hydroxymethylation at that locus.

“Accuracy” refers to the degree of conformity of a measured or calculated quantity (a test reported value) to its accurate (or true) value. Clinical accuracy relates to the proportion of true outcomes (true positives (TP) or true negatives (TN) versus misclassified outcomes (false positives (FP) or false negatives (FN), and may be stated as a sensitivity, specificity, positive predictive values (PPV) or negative predictive values (NPV), or as a likelihood, or odds ratio, among other measures.

“Performance” is a term that relates to the overall usefulness and quality of a diagnostic or prognostic test, including, among others, clinical and analytical accuracy, other analytical and process characteristics, such as use characteristics (e.g., stability, ease of use), health economic value, and relative costs of components of the test. Any of these factors may be the source of superior performance and thus usefulness of the test, and may be measured by appropriate “performance metrics,” such as AUC, time to result, shelf life, etc. as relevant.

“Clinical parameters” encompass all non-sample biomarkers of subject health status or other characteristics, such as, without limitation, lesion size; lesion location; patient age; patient weight; patient gender; patient ethnicity; family history; genetic mutations; and PD-L1 tumor staining result, which is currently used in the clinic to determine whether anti-PD-1 therapy is in order.

A “formula,” “algorithm,” or “model” is any mathematical equation, algorithmic, analytical or programmed process, or statistical technique that takes one or more continuous or categorical inputs and calculates an output value, sometimes referred to as a “probability score” or “index value.” Non-limiting examples of “formulas” include sums, ratios, and regression operators, such as coefficients or exponents, biomarker value transformations and normalizations (including, without limitation, those normalization schemes based on clinical parameters, such as gender, age, or ethnicity), rules and guidelines, statistical classification models, and neural networks trained on historical populations.

Of particular interest herein are linear and non-linear equations and statistical classification analyses to determine the correlation between hydroxymethylation levels at the biomarker loci detected in a patient sample and the patient's likelihood of having a particular type of cancer. In panel and combination construction, of particular interest are structural and syntactic statistical classification algorithms, and methods of risk index construction, utilizing pattern recognition and machine learning features, including established techniques such as cross-correlation, Principal Components Analysis (PCA), factor rotation, Logistic Regression (LogReg), Linear Discriminant Analysis (LDA), Eigengene Linear Discriminant Analysis (ELDA), Support Vector Machines (SVM), Random Forest (RF), Recursive Partitioning Tree (RPART), as well as other related decision tree classification techniques, Shrunken Centroids (SC), StepAIC, Kth-Nearest Neighbor, Boosting, Decision Trees, Neural Networks, Bayesian Networks, and Hidden Markov Models, among others. Many such algorithmic techniques have been further implemented to perform both feature (loci) selection and regularization, such as in ridge regression, lasso, and elastic net, among others. Other techniques may be used in survival and time to event hazard analysis, including Cox, Weibull, Kaplan-Meier and Greenwood models well known to those of skill in the art. Many of these techniques are useful either combined with a hydroxymethylation biomarker selection technique, such as forward selection, backwards selection, or stepwise selection, complete enumeration of all potential biomarker sets, or panels, of a given size, genetic algorithms, or they may themselves include biomarker selection methodologies. These may be coupled with information criteria, such as Akaike's Information Criterion (AIC) or Bayes Information Criterion (BIC), in order to quantify the tradeoff between additional biomarkers and model improvement, and to aid in minimizing overfit. The resulting predictive models may be validated in other studies, or cross-validated in the study they were originally trained in, using such techniques as Bootstrap, Leave-One-Out (LOO) and 10-Fold cross-validation (10-Fold CV). At various steps, false discovery rates may be estimated by value permutation according to techniques known in the art.

“Likelihood,” in the context of one embodiment of the present invention, is the probability that a patient has or does not have pancreatic cancer.

A “hydroxymethylation level” refers to the extent of hydroxymethylation within a hydroxymethylation biomarker locus. The extent of hydroxymethylation is normally measured as hydroxymethylation density, e.g., the ratio of 5hmC residues to total cytosines, both modified and unmodified, within a nucleic acid region. Other measures of hydroxymethylation density are also possible, e.g., the ratio of 5hmC residues to total nucleotides in a nucleic acid region.

A “hydroxymethylation profile” or “hydroxymethylation signature” refers to a data set that comprises the hydroxymethylation level at each of a plurality of hydroxymethylation biomarker loci that are preselected as differentially hydroxymethylated with regard to a particular disease phenotype, e.g., lung cancer, colorectal cancer, breast cancer, or the like. The hydroxymethylation profile may be a reference hydroxymethylation profile that comprises composite a hydroxymethylation profile for a population of individuals with at least one shared characteristic, as explained elsewhere herein. The hydroxymethylation profile may also be a patient hydroxymethylation signature, constructed from the measurement of hydroxymethylation levels at each of a plurality of hydroxymethylation biomarker sites.

The term “locus” as used throughout this application refers to a site on a nucleic acid molecule, wherein the nucleic acid molecule may be single-stranded or double-stranded, and further wherein an individual locus (or multiple “loci”) may be of any length, thus including a single CpG site as well as a full-length gene, or across larger features such as topologically associated domains, including when several such loci are aggregated into groups such as related sequence motifs, other homologies or functional characteristics (regardless of their adjacency or topological relationship). The loci herein may be contained within a gene body; within an annotation feature outside of the gene body, such as a promoter, an enhancer, a transcription initiation site, a transcription stop site, or a DNA binding site, or a combination thereof; or within an untranslated region, or “UTR” (including 3′UTRs and 5′UTRs).

It should be noted that some of the individual biomarkers disclosed herein, e.g., hydroxymethylation biomarkers, may not have significant individual significance in a particular evaluation, but when used in combination with one or more other types of biomarkers and, optionally, clinical parameters impacting on the detection and evaluation of a cancerous lesion become significant in discriminating as a method of the invention requires.

The term “correlate” as used herein in reference to two variables (e.g., two values, two sets of values, a value or value set and a disease state, a value or set of values and a risk associated with the disease state, or the like) indicates a tendency of the two variables to vary together. A “correlation” is a measure of the extent to which two or more variables fluctuate together. A positive correlation indicates the extent to which those variables increase or decrease in parallel. One example of a positive correlation is the relationship between a hydroxymethylation level at a hydroxymethylation biomarker locus, on the one hand, and the likelihood that a patient has cancer or a particular type of cancer, on the other. Conversely, a negative correlation would exist when the hydroxymethylation level at a hydroxymethylation biomarker locus decreases as a subject's likelihood of having cancer or a particular type of cancer decreases.

### II. Method for Identifying Cancer in a cfDNA Sample

The method of the invention relies on a combination of “base” models set forth in Table 1, previously incorporated by reference herein. Table 1 provides a list of regions identified in the sequenced cfDNA and information about how each region is used in each base model, as explained below.

Each feature name entry in the column “featurename” in Table 1 is a region in the sequenced cfDNA identified by its corresponding coordinates in the human reference genome hg38, separated by colons. More specifically, each feature name entry begins with the chromosome, then gives the location of the nucleotide termini with starting position specified first followed by the ending position, and lastly, an identifier of some sort, e.g., a gene name. For example, the feature name chr17:5111410:5112061:CpG-10567 refers to a region on chromosome 17 that begins at position 5111410 and terminates at position 5112061, with the identifier “CpG-10567.” Each entry in the column “hg38_genomic_coordinates” is the gene region to which the corresponding identified region aligns, with the hg38 coordinates provided in the same manner as above.

In the first column of Table 1, each entry under “model” is the name selected to correspond to a group of similar features that are used in one of the “base” models. As one example, the base model might include regions from promoters. Each row identified as “promoter_decontaminated_CPM_GLMNET” in the “model” column, for instance, lists a region of the genome corresponding to a promoter where the number of fragments were where the number of fragments were counted that he promoter across our cancer and control samples, then used elastic net to fit a model.

Certain feature types are only used to build models from 5hmC data obtained from 5hmC-containing cfDNA fragments using the 5hmC assay protocol described in the Example, and are identified as such in the column “assay” in Table 1. Other feature types are only used to build models from WGS data and, again, and those are designated “WGS” in the “assay” column. For example, only WGS fragments are considered in the base models “Frag_Data_2 MB_scaled_GLMNET” and “WGS_CNV_100_kb_GLMNET.”

With respect to fragment length, it should be noted that all sequenced fragments in Table 1, whether generated by the 5hmC assay or WGS, are filtered to exclude fragments outside of the 50 nt to 1000 nt size range, i.e., for all samples and libraries. However, as just noted, fragment size is taken into account as a separate feature for the base model “Frag_Data_2 MB_scaled_GLMNET,” in which fragments are counted and sorted into one of three size range bins, 50-152 nt, 153-240 nt, or 241-1000 nt; they are also specified in terms of location in one of a series of 2 Mbp windows along the genome. Accordingly, there are three rows for each 2 Mbp window, insofar as there may be fragments within each of the three size ranges contained within the same 2 Mbp window.

The column “cfdna_plasma_interaction_term” indicates whether, for that particular region in the genome, cfDNA concentration is used as a factor together with the count of fragments mapping to the region. Specifically, when that column has the value “yes,” the cpm value is multiplied by the log of 1+the concentration. This helps performance, i.e., overall prediction power, because patients with cancer tend to have higher concentrations of cfDNA in their plasma.

Each value in the “coefficient” column is used to multiply the cpm value from the sample for that region. The sum of the coefficient x the cpm values (or coefficient x cpm x log [1+conc]) gives the log odds or logit for the corresponding base model, which has a 1-1 correspondence with the p value score.

The ENSEMBLE rows show how the base model scores are combined into a final score, a probability score p between zero and 1 indicating the likelihood of pancreatic cancer.

Additional information pertaining to various aspects of the invention may be found in U.S. patent application Ser. No. 17/131,287, filed Dec. 22, 2020, for “Pancreatic Ductal Adenocarcinoma Evaluation Using Cell-Free DNA hmC Profile,” and includes methods for extending the present invention to encompass patient monitoring as well as assessment of treatment. The disclosure of that application is incorporated by reference herein in its entirety.

A representative embodiment of the present process is described in detail in the Example below.

### Example

Patient samples were collected under the clinical trial NCT03869814 case-control study, the goal of which was to employ genomics, epigenomics, and proteomics methodology to optimize a method for detecting cancer in the blood of subjects with solid tumors. The study included subjects without cancers that were followed up every six months for up to three years from blood draw.

### A. Study Design and Methods

### (i) Clinical Cohorts and Study Design:

From June 2018 to May 2022, cancer and noncancer subjects from 146 sites across the United States were recruited into a case-control study (NCT03869814). All subjects provided written informed consent, and the study was approved by the institutional review boards (IRBs) responsible at each site. The study protocol submission, IRB approval, and specimen handling across all sites were managed by several contract research organizations.

### Study Inclusion Criteria:

(1) Subjects were between 45-75 years of age at the time of enrollment;
(2) Patient fully consented;
(3) Cancer diagnosis or a high clinical suspicion for cancer, based on the participating site's and practitioner's standards of care (SOC); and
No previous history of cancer and treatment naïve at time of enrollment

### Study Exclusion Criteria:

(1) Age<45 OR >75 years of age;
(2) Any prior cancer diagnosis with or without treatment (with the exception of non-melanoma skin cancers resolved/treated >1 year prior to enrollment);
(3) Receipt of any cancer therapy including chemotherapy, radiation, palliative radiation, hormonal or naturopathic therapies;
(4) In situ carcinoma without an invasive component
(5) Any surgery requiring general anesthesia within 2 months of collection (anesthesia used in procedures such as colonoscopy and EBUS is acceptable);
(6) Dental Novocain within 1 week of sample collection;
(7) Receipt of systemic immunomodulation therapy within the prior 12 months;
(8) Current pregnancy or pregnancy within the prior 12 months;
(9) Organ transplantation;
(10) Received dialysis;
(11) Blood transfusion within the previous month;
(12) HIV/AIDS, Hepatitis A, D, or E, TB, any kind of prion disorder (e.g., CJD) or infection with other pathogens currently or within the five prior years.

The case control study was divided into 2 datasets: a training set and a validation set. The validation set included a subset of individuals with NOD. Samples for the training-set predictive logistic regression-based algorithms were obtained from subjects within the training cohort who were known to not have intraductal papillary mucinous neoplasms (IPMNs), pancreatic cysts, pancreatitis, or NOD.

### (ii) Sample Collection and Plasma Preparation:

Plasma was isolated from whole blood specimens obtained by routine venous phlebotomy at the time of enrollment. Whole blood (2×10 mL) was collected in Cell-Free DNA BCT® tubes (Streck, La Vista, NE) per manufacturer's protocol and maintained at 15-25° C. for shipment to the laboratory and processed within 72 hours of venipuncture. To separate plasma, tubes were centrifuged at 1600×g for 10 minutes, and the plasma was transferred to new tubes for further centrifugation at 16,000×g for 10 minutes. The final plasma was aliquoted for frozen storage at −80° C. before cfDNA isolation.

### (III) Cfdna Isolation:

cfDNA isolation was carried out on a liquid handler (Hamilton STAR Liquid Handling System, Reno, NV) using the MagMax™ cfDNA Isolation Kit (Thermo Fisher Scientific US, Waltham, MA) according to the manufacturer's protocol. Isolated cfDNA was quantified using Quant-iT™ PicoGreen™ (Thermo Fisher Scientific US) and stored at −20° C. until library preparation.

### (iv) Tissue Handling and Genomic DNA Isolation:

Forty-three PaC (obtained from surgeries) and 10 normal pancreatic tissue samples (two obtained as normal adjacent tumor; and eight from normal pancreas) were collected and stored in Hypo Thermosol (H) media (Sigma, St. Louis, MO). All tissue samples were obtained from surgeries. Each sample was weighed and aliquoted into sections of approximately 35 mg, homogenized in 500 μl RLT Buffer Plus using a Tissue Lyser LT (QIAGEN, Germantown, MD), and stored at −80° C. until DNA extraction. Genomic DNA was extracted using DNeasy Blood & Tissue Kit (QIAGEN, Hilden, Germany) according to the manufacturer's instructions. Genomic DNA eluates were quantified using the Qubit dsDNA High Sensitivity assay (Thermo Fisher Scientific US) and stored at −20° C. until further processing. Prior to sequencing library construction, genomic DNA was fragmented to a modal 150 base pair size using an ME220 Focused-ultrasonicator (Covaris, Woburn, MA); modal fragmented DNA sizes were verified using the 2200 TapeStation dsDNA high sensitivity assay (Agilent Technologies, Santa Clara, CA) and quantified as described earlier, prior to commencing library construction.

### (v) 5hmC Assay Enrichment:

Isolated cfDNA from a single BCT tube or DNA isolated from tissues were normalized to either 10 ng (cfDNA) or 20 ng (tissue DNA) in 96-well plates using an automated liquid handler (Beckman Coulter Life Sciences, Biomek 17, Indianapolis, IN), and were end-repaired, A-tailed, and ligated to sequencing adapters, to generate whole genome sequencing (WGS) libraries. A portion of the WGS libraries for each sample proceeded to complete library preparation, while the remainder of the WGS libraries underwent further processing to enrich for fragments containing 5hmC bases. The 5hmC enrichment was performed through a biotinylation reaction via 2-step chemistry, as has been outlined previously (1), and enriched by binding to Dynabeads M-270 coated with streptavidin (Thermo Fisher Scientific US). Following the enrichment of 5hmC fragments, both 5hmC and WGS libraries were amplified by polymerase chain reaction and normalized to 1 ng/μL using an automated liquid handler (Hamilton Microlab STARLet, Reno, NV)). After normalization, libraries of WGS and 5hmC were pooled and sequenced on the NovaSeq6000 sequencer (Illumina, Inc., San Diego, CA).

### B. Bioinformatic Analysis

### (i) Bioinformatics Pipeline and Quality Control:

Sequencing data from 5hmC and WGS were produced using NovaSeq Control Software v1.7.0 (Illumina, Inc., San Diego, CA). Raw data processing and demultiplexing were performed using bcl2fastq Conversion Software (Illumina, Inc., San Diego, CA) to generate sample specific FASTQ output. Sequencing reads were analyzed by a computational pipeline implemented as a Nextflow script, which aligns the reads to the human genome build 38 reference genome using the BWA-MEM2 algorithm. The pipeline divided the genome into functional regions identifying gene bodies, enhancers, CpG islands, CCCTC-binding factor sites, promoters, and 3-prime untranslated regions from Gencode human annotation version 31 (GRCh38.p12). Subsequently, the number of 5hmC library read pairs mapped to each region were enumerated, correcting for variation in coverage using counts per million mapped reads. In addition, feature sets incorporating copy number changes across 100 kb bins as ascertained by depth of read coverage and fragment size variation in 2 MB bins across the genome were created using the WGS data. Metrics were computed by the pipeline via Picard to assess the quality of the sequencing data. Samples passing quality control metrics were placed into the 2 datasets to be used for training and validation. The quality control failure rate for the set of validation samples was 1.94%. Noncancer samples in the training data were matched to various clinical features such as age, sex, body mass index, and smoking status.

### (ii) Pancreatic Cancer Algorithm Development:

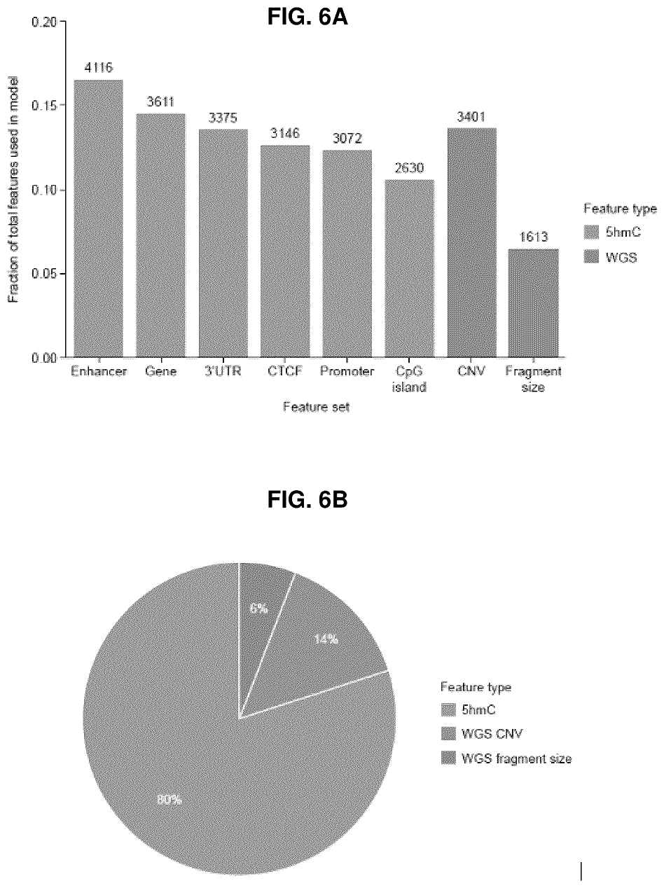

The machine-learning classification algorithm was trained as follows: each sample included in the training dataset was analyzed with the bioinformatics pipeline as described in the previous section. Elastic net logistic regression algorithms were built using the R package glmnet for each of the feature sets, with elastic net mixing ratio α and the regularization parameter λ optimized using 10-fold cross validation. After removing highly variable features, the regularization performed by elastic net further reduced the number of features and assigned coefficients to each (FIGS. 6A and 6B). Taken together, the coefficients β and the features x from the N various feature sets form an equation that calculates the log odds ratio of the individual having cancer:

$$log ( \frac{p}{1 - p} ) = {β}_{0} + {\sum }_{j = 1}^{N} {β}_{j} {\sum }_{i = 1}^{{M}_{j}} [ {β}_{i , j} {x}_{i , j} + {β}_{0 , j} ]$$

Solving the equation for p provides a probability of cancer between 0 and 1.

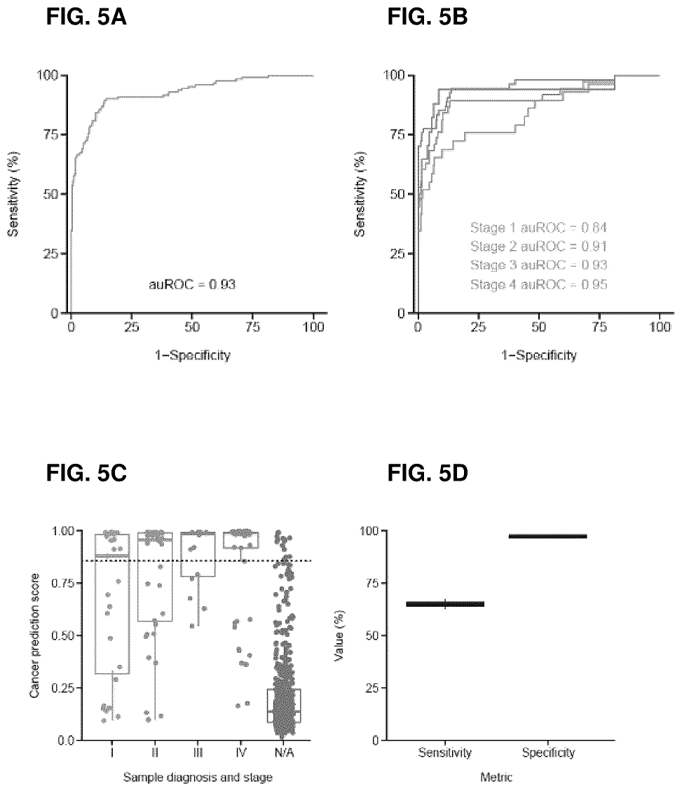

The classification probability threshold was determined by setting a threshold that resulted in 98% specificity of the noncancers in the training dataset (FIG. 5C). The final algorithm was then integrated into our automated computational pipeline as a Docker container on Amazon Web Service (Amazon, Seattle, WA). Scoring and cancer classifications were performed in a blinded fashion and subject labels were revealed after scoring was completed.

(iii) Differential Gene Representation Analysis:

For differential 5hmC gene analysis, genes that did not map to autosomes were removed. Additionally, weakly represented genes were removed by excluding those that did not have >3 counts per million reads in at least 10 samples. This filter excludes roughly 7.5% of all genes from the Consensus Coding Sequence Database. The R package “edgeR” (2) was then employed to identify fold change between PaC and noncancer for both the NOD and non-NOD (any subject without a new onset diabetes diagnosis) cohorts.

### (iv) CA 19-9 Clinical Concordance Analysis:

For 507 noncancer and 73 PaC samples, 500 μl of frozen plasma was shipped to a Clinical Laboratory Improvement Amendments certified laboratory (ARUP Laboratories, UT, USA) for the evaluation of carbohydrate antigen 19-9 (CA19-9). Samples with a CA19-9 value >37 were classified as cancer, as per standard clinical thresholds employed for CA19-9 and PaC detection.

### C. Results

### (i) Development of a Pancreatic Cancer Detection Algorithm:

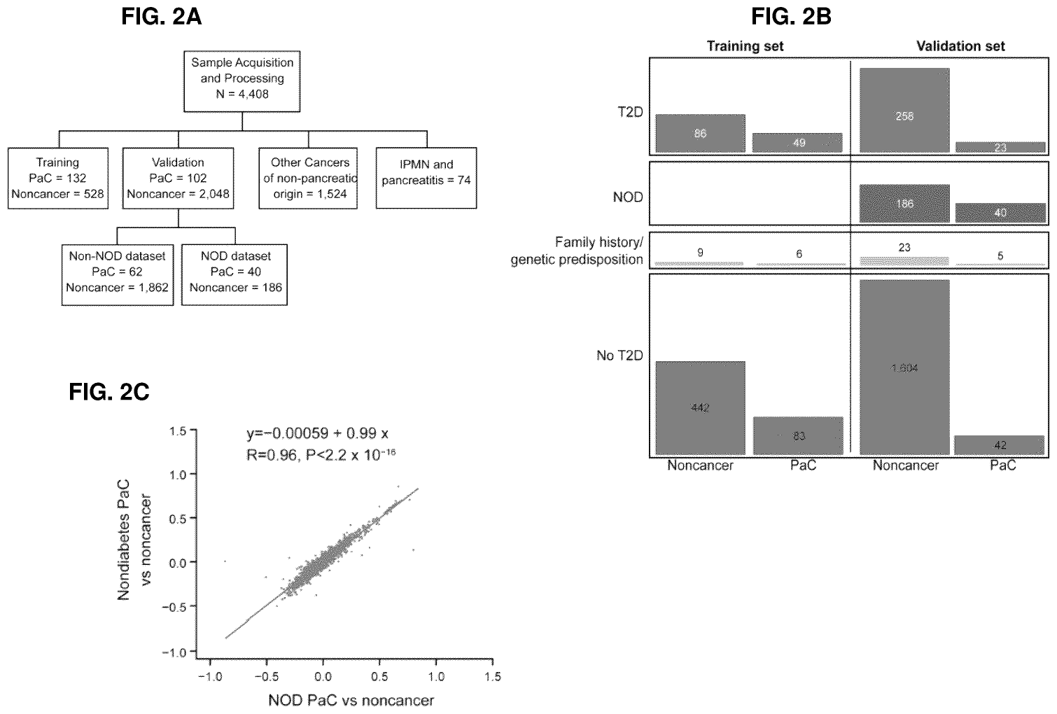

A total of 660 plasma samples (training dataset), from 132 PaC subjects and 528 noncancer subjects, were employed for the development of a PaC detection algorithm, capable of distinguishing between PaC and noncancer (FIG. 2A). Key clinical characteristics of the cohort, including body mass index, age, and other PaC risk factors such as smoking, diabetes status, family history, and genetic predisposition for PaC are reported in FIG. 2B and in Table 2:

TABLE 2

| | Training set | | Validation set | |
|---|---|---|---|---|
| Number of samples | Noncancer | PaC | Noncancer | PaC |
| Body mass index >32 kg/m² | 103 | 32 | 699 | 18 |
| Heavy smokers | 117 | 28 | 270 | 24 |
| Type 2 diabetes | 86 | 49 | 258 | 20 |
| New-onset type 2 diabetes | — | — | 186 | 40 |
| Family history/genetic disposition | 9 | 6 | 23 | 5 |

Statistical analysis of the clinical variables revealed that in the validation dataset the PaC subjects were older and had lower BMI compared to noncancer subjects (Student's t test, p<0.05). Furthermore, overall, the validation dataset had fewer males compared to the training dataset (Fisher's Exact Test, P<0.05). Also, PaC subjects in both sets showed higher proportion of former smokers while the proportion of current smokers was also higher in the validation set (Fisher's Exact Test, P<0.05).

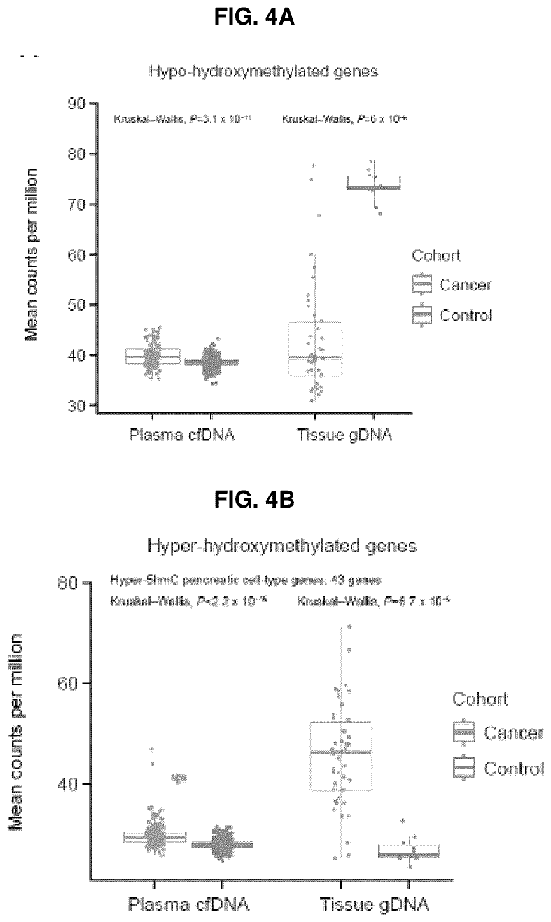

First, we evaluated whether 5hmC signals found in PaC tumor tissues could be detected in plasma cfDNA by identifying sets of genes with significant over- or under-representation by their 5hmC levels. Hence, we compared 42 PaC tumor tissue samples and 10 normal tissue samples and identified 366 genes with decreased hydroxymethylation and 43 genes with increased hydroxymethylation (FDR 0.05 and with a 1.5-fold change). These same hyper and hypo-hydroxymethylated genes were interrogated in cfDNA from PaC and noncancer subjects. Consistent trends in 5hmC representation found in tumor versus normal pancreatic tissue (Kruskal-Wallis P<3.1×10 sets (ref. 11) and P<2.2×10 (ref. 16)) for the increased and decreased hydroxymethylated gene sets, respectively (FIG. 4A and FIG. 4B). This indicates that cfDNA of subjects with PaC carries a specific and differential 5hmC profile, enabling the use of 5hmC signals to detect PaC using plasma-derived cfDNA.

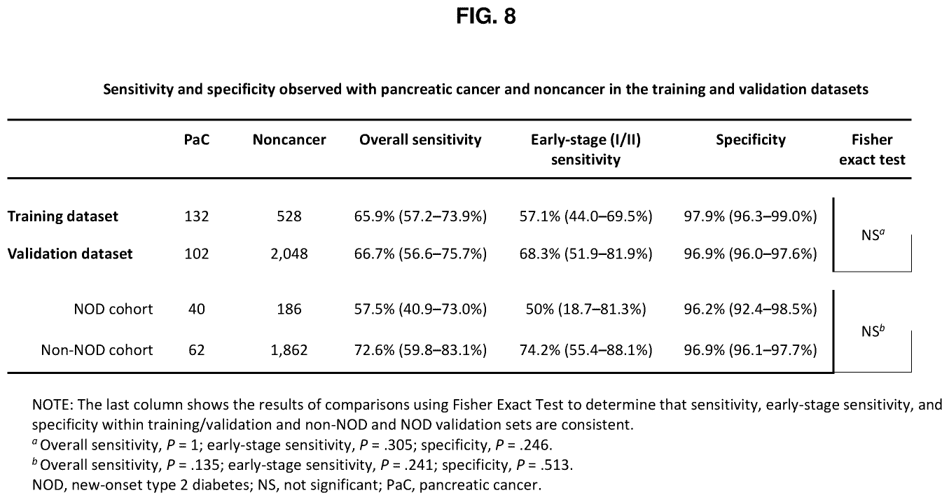

Next, we developed a specific algorithm for cfDNA PaC and, after matching for body mass index, age, and smoking status within the training cohort (Table 2), a binomial prediction algorithm was constructed using elastic net logistic regression combining predictors from both the 5hmC and WGS features. This yielded an overall performance measured by area under the Receiver Operating Characteristics (auROC) curve of 0.93 overall and 0.84-0.95 stage specific range (FIGS. 5A-5C). The feature set with the greatest contribution to the algorithm was 5hmC loci in enhancer regions, followed by 5hmC loci in gene body regions (FIG. 6A and FIG. 6B). To simulate how well the algorithm would perform on new data, the algorithm was assessed using 10-fold cross validation enabling 10% of samples to be held from the training set and instead used for validation. For each of the 10 folds, the training data were split into training and test sample partitions, in the ratio 90:10. Overall test sensitivity for this 10-fold cross validation analysis was 65.9% (95% confidence interval [CI], 57.2-73.9%), with an early-stage (stage I/II) sensitivity of 57.1% (95% CI, 44.0-69.5%) and a specificity of 97.9% (95% CI, 96.3-99.0%), as indicated in FIG. 8. To evaluate the repeatability of the 10-fold cross validation, the process was repeated 10 times using different sample fold assignments; this yielded consistent sensitivity (62.8-67.4%) and specificity (97.5-98.1%) (FIG. 5D).

### (ii) Validation of the Pancreatic Cancer Detection Algorithm:

Next, we evaluated the performance of the PaC detection test in a separate set of samples blindly and independently processed from the training set. The validation dataset included 2,150 subjects consisting of 102 PaC and 2,048 noncancer subjects. A sensitivity of 68.3% (95 CI, 51.9-81.9%) was observed in early-stage disease (stage I/II samples combined), with an overall sensitivity of 66.7% (95 CI, 56.6-75.7%) and a specificity of 96.9% (95% Cl, 96.0-97.6%) (FIG. 8). Statistical tests determined that there was no significant difference in the validation set performance and that of the training set and between NOD and non-NOD subjects.

In addition to the validation dataset, a set of 74 noncancer samples with other pancreatic lesions (excluding PaC) were tested; these included 40 subjects with IPMNs, 27 with pancreatitis, and 7 with both conditions. The algorithm predicted PaC in 26 of 47 (55.3%) subjects with IPMNs and 14 of 34 (41.2%) subjects with pancreatitis. Of note, the majority of samples from subjects with IPMN (18/26 [69.2%]) that were classified as PaC had moderate to high dysplasia and 1 case had PanIN-2 disease. In contrast, only 14.3% (3/21) of the IPMNs classified as “not detected” by our test had moderate to high dysplasia.

Additionally, we evaluated the algorithm against 1,524 subjects across 11 different diseases with a cancer diagnosis other than PaC (FIG. 2A and Table 3).

TABLE 3
Detection rates for nonpancreatic cancers:

| | | Number of | | |
|---|---|---|---|---|
| | Number of | samples | Positive tests, % | |
| Cancer Type | samples | detected | (95% CI) | |
| Bladder cancer | 33 | 6 | 18.2 | (6.98-35.5) |
| Breast cancer | 400 | 57 | 14.2 | (11.0-18.1) |
| Colorectal cancer | 198 | 40 | 20.2 | (14.8-26.5) |
| Esophageal cancer | 58 | 15 | 25.9 | (15.3-39) |
| Gastric cancer | 33 | 9 | 27.3 | (13.3-45.5) |
| Kidney cancer | 196 | 36 | 18.4 | (13.2-24.5) |
| Liver cancer* | 29 | 16 | 55.2 | (35.7-73.6) |
| Lung cancer | 210 | 61 | 29.0 | (23.0-35.7) |
| Ovarian cancer | 36 | 21 | 58.3 | (40.8-74.5) |
| Prostate cancer | 239 | 11 | 4.6 | (2.3-8.1) |
| Uterine cancer | 92 | 14 | 15.2 | (8.6-24.2) |
| Total nonpancreatic cancer | 1,524 | 286 | 18.8 | (16.8-20.8) |

CI, confidence interval

Subjects with stage IV cancers were excluded to avoid a detection of a signal arising from occult metastasis in the pancreas. The detection rate for nonpancreatic cancer samples was determined for bladder, breast, colorectal, esophageal, gastric, kidney, liver, lung, ovarian, prostate, and uterine cancers in the independent validation set. Liver and ovarian cancers had the highest rates of detection, at 55.2% and 58.3%, respectively. Of note, of the liver cancers detected by algorithm, three had pancreatobiliary origin, four were intrahepatic bile duct carcinoma, and one was a cholangiocarcinoma which all share pancreatic tissue features. The gastrointestinal cancers (colorectal, esophageal, and gastric) had a moderate rate of detection (20.2%, 25.9%, and 27.3%, respectively), compared with prostate and breast cancers (4.6% and 14.2%, respectively), for example.

(iii) Validation of the Pancreatic Cancer Detection in a High-Risk Population:

The validation dataset contains 2,150 subjects inclusive of individuals with high-risk clinical conditions for PaC, such as a family history, genetic predisposition, long-standing type 2 diabetes (>3 years from diabetes diagnosis) and NOD (diagnosed with type 2 diabetes within 3 years from diagnosis) (FIG. 2B and Table 2). As indicated in FIG. 2B, the training set did not include subjects with NOD.

We carried out a separate evaluation of the performance in subjects with NOD, the larger group of subjects with high risk for PaC (6-8 times relative risk) within the validation set. When comparing the performance in subjects with NOD versus those without NOD in the validation set, no significant difference was found (Fisher Exact Test, P>0.05); sensitivity was 57.5% (95% CI, 40.9-73.0%) and 72.6 (95% CI, 59.8-83.1%) for NOD and non-NOD, respectively. Specificity was also determined to be not significantly different and was 96.2% (95% CI, 92.4-98.5%) and 96.9% (95% CI, 96.1-97.7%) for NOD and non-NOD, respectively (FIG. 8). Further, we evaluated the performance of pancreatic cancer detection algorithm in other high-risk groups and observed a sensitivity of 88.9% (95% CI 51.8%, 99.7%) and a specificity of 94.2% (95% CI 90.1%, 97%) for long term diabetics, 71.4% (95% CI 47.8%, 88.7%) sensitivity and 97.2% (95% CI 94.3%, 98.9%) specificity for current smokers. Sensitivity was lower in subjects with BMI>32, 41.2% (95% CI 18.4%, 67.1%) while specificity was maintained, 96% (95% CI 94.2%, 97.3%). Together, these data suggest that the algorithm detects a PaC signal independently of clinical risk status.

To verify this observation, gene-based differential changes in 5hmC profile found in cancer versus noncancer were compared in a pairwise manner between subjects without diabetes and those with NOD. A high level of correlation was shown (r=0.96; P<2.2×10⁻¹⁶), supporting the findings that the gene-based differential 5hmC profiles are consistent between cancer and noncancer and are not impacted by type 2 diabetes status (FIG. 2C).

(iv) Performance Comparison with CA19-9 Biomarker:

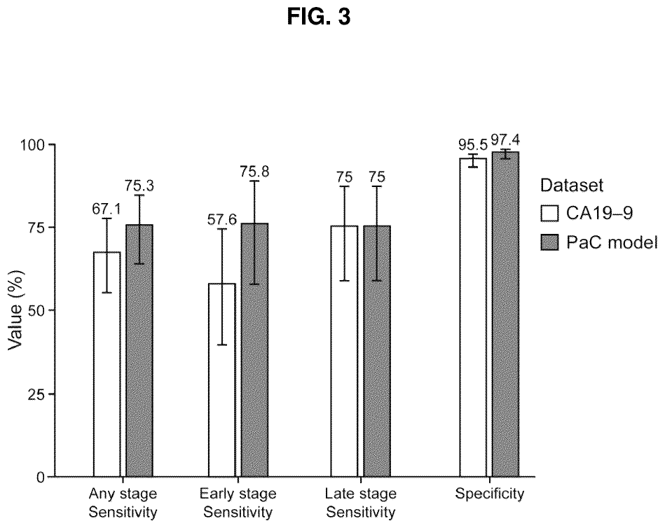

CA19-9 is the most used biomarker for diagnosis and management of PaC (25); therefore, we evaluated the agreement between CA19-9 and the current PaC detection test. CA19-9 analysis was performed on 73 PaC and 507 noncancers and compared with predictions made using the PaC detection algorithm. The PaC detection performance in early-stage cancer was higher using the current PaC detection test than the CA19-9, with a 75.8% (95% CI, 57.7-88.9%) sensitivity and 97.4% (95% CI, 95.7-98.6%) specificity, compared with 57.6% (95% CI, 39.2-74.5%) sensitivity and 95.5% (95% CI, 93.3-97.1%) specificity when using CA19-9 alone (FIG. 3). Notably, the false positive rate of CA19-9 was nearly double that of the PaC detection test, at 4.5% (95% CI, 2.9-6.7%) and 2.6% (95% CI, 1.4-4.3%), respectively.

### D. Significance of Results

The data from our study provides evidence that differential epigenomic signals identified by 5hmC measurement found in pancreatic tumor tissue are also found in cfDNA from distinct patients. Employing cfDNA derived 5hmC and genomic signals enables the construction of a robust pancreatic cancer detector as shown by a stable performance that was similar in the training (n=660) and validation (n=2,150) datasets. The large validation dataset exhibited a cancer incidence of ˜5% which approaches the clinical incidence of cancer in high-risk populations of ˜1% (e.g., NOD, family history and mutations). The performance of PaC detection was maintained at 66.7% sensitivity and 96.9% specificity compared with the training dataset, and also retained in samples from patients with early-stage PaC (Stage I or II; 68.3% sensitivity).

The PaC signal detector employing epigenomic and genomic signal outperforms CA19-9 measures on analytically matched cohorts, particularly for early-stage PaC.

CA19-9 is the only biomarker routinely used in the management of PaC; however, due to its poor sensitivity in early-stage disease, its lack of expression in individuals with a Lewis-negative genotype, and its elevation in many other benign and malignant diseases, it has been used sparingly in early-detection protocols (22). We compared the performance of CA19-9 and our PaC detection test in a subset of patients. Our PaC detection test outperformed early-stage performance of CA19-9, with a 75.8% (95% CI, 57.7-88.9%) sensitivity and 97.4% (95% CI, 95.7-98.6%) specificity, compared with 57.6% (95% CI, 39.2-74.5%) sensitivity and 95.5% (95% CI, 93.3-97.1%) specificity for CA19-9 alone. This supports the indication for our test in early disease detection.

Targeted, routine clinical assessment is only recommended for individuals whose lifetime risk of developing pancreatic ductal adenocarcinoma is higher than 5%, which includes those with familial history and particular genetic syndromes, or subjects with mucinous cystic lesions of the pancreas (15). In addition, another high-risk group includes patients with NOD, who are at a 6- to 8-fold increased risk of developing the disease (17,19). A previous study has demonstrated that the 3-year cumulative incidence rate of PaC in subjects with NOD is 0.85% (19). Other studies have reported variable incidence of Pac in NOD subjects ranging from 0.3% (23) to 3% (24) to 10% (25). Variability in the reported incidence appears to be impacted population characteristics distribution (such as age, gender and ethnicity) or size of the study.

Overall, in the United States there are nearly 1.4 million individuals diagnosed with NOD annually; about 900,000 are age 50 and above, and up to 1% will develop PaC within 3 years of their type 2 diabetes diagnosis advocating that this NOD group would benefit from early PaC detection. In our validation cohort, we demonstrated the detection of PaC in a subpopulation of subjects with NOD with the same level of performance as shown in the full dataset that included subjects with type 2 diabetes and subjects without diabetes. Of note, the training set did not include subjects with NOD, supporting the evidence that the algorithm is detecting PaC independent of type 2 diabetes status. Further, the close correlation of 5hmC signatures of PaC with and without NOD (see FIG. 2C) indicates that the PaC biology detected by 5hmC is similar in the NOD and non-NOD populations and supports the use of this test in subjects with NOD.

IPMNs, which are characterized by intraductal papillary proliferation of mucin-producing epithelial cells, are also well known as precursor lesions for PaC26, albeit that a low rate of patient with IPMNs progress to PaC. There is evidence that epigenetic changes are present in IPMNs and increase with progression to PaC (27). Of note in this study is that the PaC algorithm can detect subjects with IPMNs displaying moderate to high levels of dysplasia. Further development, using 5hmC signatures in relevant cohorts, will enable us to train the algorithm to detect which precursor IPMNs result in PaC suggesting that they need to be closely monitored (28).

In conclusion, a precise, scalable, and efficient assay has now been provided that requires only 10 ng of cfDNA. The combination of the 5hmC assay and associated PaC-specific detection algorithm enables the effective measurement of cancer presence in individuals at high risk for PaC, thereby offering a molecular tool for earlier detection and timely intervention.

1. A method for detecting the likelihood of pancreatic cancer in a patient from a blood sample obtained from the patient, comprising:
(a) extracting a cell-free DNA (cfDNA) sample from the blood sample;
(b) counting 5-hydroxymethylcytosine (5hmC)-containing DNA fragments in the cfDNA sample to give a total 5hmC fragment count;
(c) counting 5hmC-containing DNA fragments that map to a first genomic location, giving a first genomic location 5hmC fragment count;
(d) counting 5hmC-containing DNA fragments that map to a second genomic location, giving a second genomic location 5hmC fragment count;
(e) optionally carrying out (d) to count 5hmC-containing DNA fragments that map to at least one additional genomic location, giving at least one additional genomic 5hmC fragment count;
(f) normalizing the 5hmC fragment counts obtained in (b) through (e) to give normalized 5hmC fragment counts;
(g) scoring the normalized 5hmC fragment counts using each of a plurality of base models each comprising a penalized logistic regression fit, wherein one score is generated for each base model by summing the product of a determined correlation coefficient times the normalized 5hmC fragment count, thereby providing a plurality of base model scores; and
(h) inputting the plurality of base model scores into an ensemble model to generate a final score in the form of a probability score p between zero and 1 indicating the likelihood of pancreatic cancer in the patient.

2. The method of claim 1, further including, in addition to the 5hmC-containing DNA fragment counts, incorporation of at least one additional feature type in the ensemble model of (h).

3. The method of claim 2, wherein the at least one additional feature type comprises WGS fragment counts in each of a series of windows along the genome and/or WGS fragment counts in each of a set of size range bins.

4. The method of claim 2, wherein the at least one additional feature type comprises copy number variation.

5. The method of claim 2, wherein the at least one additional feature type comprises epigenetic features other than 5hmC.

6. The method of claim 2, wherein the at least one additional feature type comprises CA 19-9 level.

7. The method of claim 2, wherein the at least one additional feature type comprises a histone marker.

8. The method of claim 2, wherein the at least one additional feature type comprises cfDNA concentration in the blood sample.

9. The method of claim 2, wherein the at least one additional feature type comprises a patient specific clinical parameter that correlates with the risk of pancreatic cancer.

10. A method for detecting the likelihood of pancreatic cancer in a patient from a blood sample obtained from the patient, comprising:
(a) extracting a cell-free DNA (cfDNA) sample from the blood sample;
(b) counting 5-hydroxymethylcytosine (5hmC)-containing DNA fragments in the cfDNA sample to give a total 5hmC fragment count;
(c) determining 5hmC-containing fragment counts in CTCF-binding regions, annotated enhancer regions, annotated gene body regions, and annotated 3′-UTR genomic regions, to provide individual location 5hmC fragment counts;
(d) determining WGS fragment counts in each of a series of genomic windows along the genome and/or WGS fragment counts in each of a set of size range bins;
(e) determining WGS fragment counts in specifically sized genomic regions for determination of copy number variation (CNV); (f) determining cfDNA concentration in the blood sample;
(f) normalizing the fragment counts obtained in (b) through (e) to give normalized fragment counts;
(g) scoring the normalized fragment counts using each of a plurality of base models each comprising a penalized logistic regression fit, wherein one score is generated for each base model by summing the product of a determined correlation coefficient times the normalized fragment count, thereby providing a plurality of base model scores; and
(h) inputting the plurality of base model scores into an ensemble model to generate a final score in the form of a probability score p between zero and 1 indicating the likelihood of pancreatic cancer in the patient.

11. The method of claim 10, wherein the method further comprises incorporating at least one additional feature type in the ensemble model of (h).

12. The method of claim 11, wherein the at least one additional feature type comprises cfDNA concentration in the blood sample.

13. A method for monitoring changes in a patient's pancreatic cancer state, comprising repeating the process of claim 1 at intervals throughout an extended monitoring period and determining differences in the probability score over time.
